(19) United States
(12) Patent Application Publication
(10) Pub. No.: US 2011/0288141 A1
(43) Pub. Date: Nov. 24, 2011

(54) LOCAL TOPICAL ADMINISTRATION FORMULATIONS CONTAINING FIPRONIL

(75) Inventors: Keith A. Freehauf, Summit, NJ (US); Chen-Chao Wang, Summit, NJ (US); Peter Andrew O'neill, Seven Hills (AU)

(73) Assignee: INTERVET INC. AND INTERVET INTERNATIONAL B.V., Boxmeer (NL)

(21) Appl. No.: 12/674,614

(22) PCT Filed: Aug. 29, 2008
(86) PCT No.: PCT/EP2008/061394

§ 371 Date: Jul. 21, 2011

Related U.S. Application Data
(60) Provisional application No. 60/969,128, filed on Aug. 30, 2007.

(51) Int. Cl. A61K 31/415; A61P 33/14

(52) U.S. Cl. 514/404

(57) ABSTRACT

The present invention provides formulations and methods useful in eradicating ectoparasites on a domestic animal, decreasing the number of ectoparasites on a domestic animal, and/or preventing ectoparasite infestation on a domestic animal, using a local topical formulation comprising fiproniland a veterinarily acceptable carrier.

### BACKGROUND OF THE INVENTION

Animals are often susceptible to infestations by ectoparasites (e.g. flies, ticks, mites and lice), and infections by endoparasites (e.g. filariae and intestinal roundworms). Domesticated animals, such as cats and dogs, are often infested with one or more of the following ectoparasites: cat and dog fleas (Ctenocephalides felis, Ctenocephalides canis, and the like), ticks (Rhipicephalus spp., Ixodes spp., Dermacentor spp., Amblyoma spp. and the like), and mites (Demodex spp., Sarcoptes scabei., Otodectes cynotis. and the like).

Fleas are a particular problem because not only do they adversely affect the health of the animal or human, but they also cause a great deal of psychological stress. Moreover, fleas are also vectors of pathogenic agents in animals, such as dog tapeworm (Dipylidium caninum), including humans.

Similarly, ticks are also harmful to the physical and psychological health of the animal or human. However, the most serious problem associated with ticks is that they are the vector of pathogenic agents, agents which cause diseases in both humans and animal. Major diseases which are caused by ticks include borrelioses (Lyme disease caused by Borrelia burgdorfen), babesioses (or piroplasmoses caused by Babesia sp.) and rickettsioses (also known as Rocky Mountain spotted fever). Ticks also release toxins which cause inflammation or paralysis in the host. Occasionally, these toxins are fatal to the host. Moreover, mites are particularly difficult to combat since there are very few active substances which act on these parasites and they require frequent treatment.

Likewise, farm animals are also susceptible to parasite infestations. For example, cattle are affected by a large number of parasites. A parasite which is very prevalent among farm animals is the tick genus Boophilus, especially B. microplus (cattle tick), B. decoloratus and B. anulatus. Ticks, such as Boophilus microplus, are particularly difficult to control because they live in the pasture where the farm animals graze. Other important parasites of cattle include the myiases-producing flies such as Dermatobia hominis (known as Berne in Brazil) and Cochlyomia hominivorax (screwworm) whose larvae infest the tissue of the host animal. Additionally, the species Lucilia sericata (greenbottles), Lucilia cuprina (damage caused by this fly is commonly known as blowfly strike in Australia, New Zealand and South Africa) are important causes of myiases in sheep. Insects whose adult stage constitutes the parasite include: Haematobia irritans (horn fly); lice such as Linognathus vituli, etc.; and mites such as Sarcoptes scabiei and Psoroptes ovis. The above list is not exhaustive and other ectoparasites are well known in the art to be harmful to animals and humans. These include, for example migrating dipterous larvae such as Hypoderma spp. and Oestrus ovis.

Control of ectoparasites on domestic animals has been attempted using flea collars containing various insecticides. The ectoparasites, however, remain present in the general vicinity of the animal, such as within the house of a pet owner. The eradication of ectoparasites within the animal environment is difficult unless the environment is permanently covered in an insecticidal substance, in which case toxicity and reinfestation are problematic. Thus, there is a need in the art for persistent and effective agents for eradication of ectoparasites on a domestic animal in order to reduce the periodicity and the cost of anti-ectoparasite agents. The present invention meets these and other needs in the art.

### BRIEF SUMMARY OF THE INVENTION

The present invention provides improved fipronil formulations useful in controlling ectoparasites on a domestic animal. In some embodiments, the fipronil formulation is a local topical formulation comprising an ectoparasitically effective amount of fipronil and a veterinarily acceptable carrier, in the absence of an effective amount of a fipronil crystallization inhibitor, as defined herein. In other embodiments, the local topical administration formulation consists essentially of an ectoparasitically effective amount of fipronil and a veterinarily acceptable carrier. The formulation will typically comprise fipronil at a concentration of about 10% (w/v). The veterinarily acceptable carrier can be dipropylene glycol monomethyl ether, propylene carbonate or N,N dimethylacetamide. The formulation may further comprise additional components such as a second active ingredient, a colorant, an antioxidant, a light stabilizer, or a combination thereof.

The present invention also provides methods for control of an ectoparasite on a domestic animal. The methods comprise topically applying the formulations of the invention to a localized region having a surface area of less than or equal to 10% (e.g., less than 5% or less than 2%) of the total surface area of the domestic animal. The formulation can be applied as often as needed, usually no more than twice per week.

The methods and formulations of the invention can be used to control a variety of ectoparasites, including fleas, ticks, flies, or lice on domestic animals. The domestic animal can be a mammal, such as a canine or feline mammal.

### DETAILED DESCRIPTION OF THE INVENTION

### I. Definitions

As used herein the term “fipronil” refers to a member of a group of phenylpyrazole pesticide compounds disclosed in, for example, U.S. Pat. Nos. 6,096,329, 6,395,765, and 6,716,442, all of which are incorporated herein by reference. Fipronil has the formula 1-[2,6-Cl₂4-CF₃ phenyl]3-CN 4-[SO—CF₃]5-NH₂ pyrazole. Fipronil and related phenylpyrazole compounds may be prepared according to processes described in patent applications WO-A-87/3781, 93/6089 and 94/21606 or European patent application EP-A-295,117, all of which are incorporated herein by reference.

As used herein, a “local topical formulation” is a fluid formulation including an amount of fipronil and a veterinarily acceptable carrier that is effective in controlling ectoparasites on a domestic animal (i.e., eradicating, decreasing the number, and/or preventing ectoparasite infestation on a domestic animal) when applied to less than or equal to 10% of the total surface area of a domestic animal. The “local topical formulation” may be referred to herein as “the formulation of the present invention.” A “fluid formulation” includes, for example, liquid formulations such as pour-on formulations, spot-on formulations and spray-on formulations which may be in the form of solutions, emulsions (oil-in-water or water-in-oil), suspoemulsions, microemulsions, suspensions (aqueous or non-aqueous), oils, creams and ointments. A “fluid formulation” may also include dusts, water dispersible granules, wettable powders and aerosols. The “fluid formulation” may be ready-to-use or require preparation such as dilution with water prior to use.

As used herein the term “persistent efficacy,” means that a formulation of the present invention maintains the ability to control ectoparasites over a specified period of time or conditions; for example, throughout a given number of aqueous washes, or over a given number of days, weeks or months. In some embodiments, efficacy is sufficiently persistent such that no more than a 20%, 10%, or 5% decrease in efficacy is seen after a single treatment. In this context the term “efficacy” refers to the ability of a formulation to control ectoparasite infestation.

As used herein, the terms “spot-on” and “pour-on” refer to formulations applied to a localized region or regions on an animal having a cumulative surface area of less than or equal to 10% of the total surface area of the animal, and also to the method of applying a composition to a localized surface area of an animal wherein said localized area or areas cumulatively comprise less than or equal to 10% of the total surface area of the animal.

As used herein, the term “veterinarily acceptable” refers to ingredients, compositions or methods of treatment that do not cause significant adverse reactions in or on a domestic animal, and additionally do not pose a danger of human toxicity or other adverse reactions in the surrounding environment of such animal in situations where humans may be exposed to such environment.

The term “veterinarily acceptable carrier,” as used herein, refers to solvents and/or emulsions in which fipronil is soluble and that do not cause significant adverse reactions in a domestic animal.

As used herein, the words “comprises” or “comprising” are intended as open-ended transition phrases meaning the inclusion of the named elements, but not necessarily excluding other unnamed elements.

The phrases “consists essentially of” and “consisting essentially of” are intended to mean the exclusion of additional components and/or agents that have a material effect on the basic properties of the formulations of the invention. In particular, an example of a material effect for purposes of this invention is the formation of fipronil crystals. Thus, the presence of an effective amount of an “agent that inhibits the crystallization of fipronil” or a “fipronil crystallization inhibitor” (as defined below) is not included when the term “consists essentially of” or “consisting essentially of” is employed, unless that component and/or agent is explicitly included in that formulation. On the other hand, additional components and/or agents that provide non-material effects (as defined herein) to the formulation such as, antioxidants, colorizing agents, light stabilizers, and the like may be included.

The phrases “consisting of” or “consists of” are intended as a transition meaning the exclusion of all but the recited elements with the exception of only minor traces of impurities.

In some embodiments, the local topical formulation will be prepared without an effective amount of a fipronil crystallization inhibitor or an agent that inhibits crystallization of fipronil. As used herein, the term “fipronil crystallization inhibitor” refers to one or more of the following components or agents: triacetin, polyvinylpyrrolidone, polyvinyl alcohols, copolymers of vinyl acetate and vinylpyrrolidone, polyethylene glycols, polyethoxylated sorbitan esters; lecithin, acrylic derivatives such as methacrylates and others, anionic surfactants such as alkali metal stearates, especially of sodium, of potassium or of ammonium; calcium stearate; triethanolamine stearate; sodium abietate; alkylsulphates, especially sodium laurylsulphate and sodium cetylsulphate; sodium dodecylbenzenesulphonate, sodium dioctylsulphosuccinate; fatty acids, especially those derived from copra oil, cationic surfactants such as water-soluble quaternary ammonium salts of formula N+R′R″R′″R″″, Y− in which the R radicals are optionally hydroxylated hydrocarbon radicals, and Y− is an anion of a strong acid, such as halide, sulphate and sulphonate anions; cetyltrimethylammonium bromide is among the cationic surfactants utilizable, the amine salts of formula N+R′R″R′″ in which the R radicals are optionally hydroxylated hydrocarbon radicals; octadecylamine hydrochloride is among the cationic surfactants utilizable, the non-ionic surfactants such as optionally polyethoxylated sorbitan esters, in particular Polysorbate 80, polyethoxylated alkyl ethers; polyethylene glycol stearate, polyethoxylated castor oil derivatives, polyglycerol esters, polyethoxylated fatty alcohols, polyethoxylated fatty acids, copolymers of ethylene oxide and propylene oxide, amphoteric surfactants such as substituted lauryl betaine compounds.

An “effective amount” of a fipronil crystallization inhibitor or an agent that inhibits crystallization of fipronil is the amount which substantially prevents formation of fipronil crystals or precipitates in a composition (comprising fipronil and a veterinarily acceptable carrier or solvent) that would otherwise form such crystals or precipitates. An effective amount will, of course, depend upon the particular formulation and fipronil crystallization inhibitor or agent that inhibits crystallization of fipronil. Generally, the inhibitor or agent will be present in an amount from 1 to 60% (W/V), from 5 to 50% (W/V), or from 10 to 40% (W/V). In specific embodiments of the invention, the addition of one or more: active ingredients, veterinarily acceptable solvents, and/or veterinarily acceptable carriers to a formulation of an ectoparasitically effective amount of fipronil in a veterinarily acceptable carrier is not considered an addition of an agent that inhibits the crystallization of fipronil, as used herein, even when this addition results in an inhibition of the crystallization of fipronil in the formulation.

The presence of an effective amount of an agent that inhibits crystallization of fipronil or a fipronil crystallization inhibitor may be tested using assays commonly known in the art. For example, one such assay used for testing for the formation of fipronil crystals in a formulation comprising fipronil and a veterinarily acceptable carrier (with or without the crystallization inhibitor) is carried out as follows: 20 μls of the composition are placed on a glass slide at 20° C. and allowed to dry for 24 hours. After 24 hours, the slide is observed with the naked eye to determine whether substantial crystallization is present. Formulations (with or without crystallization inhibitors) can also be tested for the ability to prevent crystallization according to methods well known in the art. Alternatively, the formulation (with or without a crystallization inhibitor) is applied to a domestic animal at room temperature, after which the animal is observed with the naked eye to determine whether substantial crystallization is present. Thus, in some embodiments, once deposited, the formulation diffuses, in particular over the animal's body, and then dries without crystallizing or modifying the appearance (in particular absence of any whitish deposit or dusty appearance) or the feel of the fur.

An “ectoparasitically effective amount” is an amount effective to control ectoparasites on a domestic animal. The term “controlling ectoparasites” refers to eradicating or decreasing the number of ectoparasites on an animal, and/or preventing ectoparasite infestation on the animal (e.g. eliminate and/or prevent infestations of ectoparasites such as adult fleas).

As used herein, the term “domestic animal” includes any animal that is kept by humans as a companion animal, pet, working animal or as livestock for food, fur, leather, wool or other animal product; or an animal that is found in association with humans such that control of ectoparasites on such animal is desirable. Common domestic animals in which the present invention will be particularly useful include a cow, horse, ass, pig, camel, bird, dog, cat, deer, sheep, or goat.

### II. Formulations and Methods for Targeting Ectoparasites on Domestic Animals

In one aspect, the present invention provides compositions and methods for controlling ectoparasites on an animal. The methods include topically applying to a localized region having a surface area of less than or equal to 10% of the total surface area of the domestic animal an ectoparasitically effective amount of a local topical formulation comprising fipronil and a veterinarily acceptable carrier.

Generally, formulations of the present invention are designed to dry without visible crystallization. In some embodiments, this goal can be achieved in the absence of an effective amount of a crystallization inhibitor. Carriers useful in such formulations include veterinarily acceptable solvents such as dipropylene glycol monomethyl ether, propylene carbonate, and N,N dimethylacetamide.

A variety of veterinarily acceptable solvents are useful in the present invention. A “veterinarily acceptable solvent,” as used herein, is a solvent that is non-toxic when topically applied to a domestic animal and is capable of sufficiently solvating fipronil to form a solution. For example, veterinarily acceptable solvents of the present invention do not cause rashes or inflammation of the dermal layer on a domestic animal. The veterinarily acceptable solvent is typically not easily ignited, or if ignited, does not burn rapidly. In some embodiments, the veterinarily acceptable solvent is non-flammable (i.e., has a flash point above the required temperature). The veterinarily acceptable solvent may also appear non-greasy after applying to said domestic animal.

In some embodiments the veterinarily acceptable solvent has a dielectric constant of between 0 and 80, 0 and 40, between 0 and 20, or between 0 and 10.

Suitable veterinarily acceptable solvents include dipropylene glycol monomethyl ether, N,N-dimethylacetamide, propylene carbonate, propylene glycol, diethylene glycol monobutyl ether, glycerin triacetate, triethyl citrate, acetyltriethyl citrate, acetyltributyl citrate, diethylene glycol monomethyl ether, diethylene glycol monoethyl ether, benzyl benzoate, ethylene glycol monobutyl ether, ethyl lactate, ethylene glycol monobutyl ether acetate, isopropyl alcohol, benzyl alcohol, ethyl acetoacetate, 2-pyrrolidinone, dimethyl isosorbide, diacetone alcohol, tetrahydrofurfuryl alcohol, propylene glycol monomethyl ether, ethanol, diethyl phthalate, heptyl acetate, methyl caprylate/caprate, N,N-dimethylcaprylamide, pentyl acetate, hexyl acetate, cyclohexyl acetate, ethylene glycol diacetate, methoxypropyl acetate, furfuryl alcohol, dibutyl phthalate, N-methylpyrrolidinone, glycerol formal, methyl salicylate, cinnamaldehyde, dimethyl sulfoxide, and cyclohexanone.

In some embodiments, other components are included in the formulation. The other component may be a second active ingredient, for example, a pesticide. Useful pesticides include insect growth regulators, antibiotic pesticides, botanical pesticides, organophosphate pesticides, carbamate pesticides, organochlorine pesticides, pyrethroid pesticides, formamidine pesticides, semicarbazone pesticides, neonicotinoid pesticides, copper-containing pesticides, anthelmintic agents, benzimidazole pesticides, salicylanilide pesticides, substituted phenol pesticides, pyrimidine pesticides, macrocyclic lactone pesticides, and imidazothiazole pesticides.

In some embodiments, the antibiotic pesticide is Bacillus thuringensis toxin. The botanical pesticides may be d-limonene, nicotine, ryania or pyrethrins. The organophosphate pesticide may be dicrotophos, terbufos, dimethoate, diazinon, disulfoton, trichlorfon, azinphos-methyl, chlorpyrifos, malathion, oxydemeton-methyl, methamidophos, acephate, ethyl parathion, methyl parathion, mevinphos, phorate, carbofenthion, phosalone, naphthalophos or pyraclofos. The carbamate pesticide may be carbaryl, carbofuran, aldicarb, or carbofuran. The organochlorine pesticide is methoxychlor, dicofol or a cyclodiene such as endosulfan. The pyrethroid pesticide may be allethrin, resmethrin, permethrin, deltamethrin cypermethrin esfenvalerate, fenvalerate, lambda-cyhalothrin, cyfluthrin or tralomethrin. The formamidine may be amitraz. The semicarbazone may be metaflumizone. The neonicotinoid pesticide may be imidacloprid, nitenpyram or dinotefuran. The copper-containing pesticide may be copper (II) hydroxide, or copper oxychloride sulfate (i.e. (Cu2Cl(OH)3) mixed with (Cu4(OH)6(SO4)). The anthelmintic agent may be a macrocyclic lactone such as an avermectin (e.g., ivermectin, moxidectin) or a milbemycin (e.g. milbemycin oxime). The benzimidazole pesticide may be albendazole or triclabendazole. The salicylanilide pesticide may be closantel or oxyclozanide. The imidazothiazole pesticide is levamisole. The pyrimidine pesticide may be pyrantel. The substituted phenol pesticide may be nitroxynil.

In some embodiments, the pesticide is sulfur, KT-199 (an antihelminthic antibiotic), or praziquantel.

The insect growth regulator may be a chitin synthesis inhibitor or a juvenile growth hormone mimic. In certain embodiments, the insect growth regulator is azadirachtin, diofenolan, fenoxycarb, hydroprene, kinoprene, methoprene, pyriproxyfen, tetrahydroazadirachtin, chlorfluazuron, cyromazine, diflubenzuron, fluazuron, flucycloxuron, flufenoxuron, hexaflumuron, lufenuron, novaluron, tebufenozide, teflubenzuron, and triflumuron.

The formulations of the present invention may also include additional agents or adjuvants, such as co-solvents, colorants, spreading agents, antioxidants, light stabilizers and/or tackifiers. Colorants are all colorants which are licensed for use on animals and which can be dissolved or suspended.

In some embodiments, an antioxidant is included. Useful antioxidants include, for example, butylhydroxyanisole, butylhydroxytoluene, ascorbic acid, sulphites, metabisulphites, or thiosulphates (e.g. sodium thiosulphate, sodium metabisulphite, potassium metabisulphite, etc.), propyl gallate, and/or tocopherol, or a mixture of not more than two of these agents.

Examples of light stabilizers are substances from the benzophenone class, or novantisolic acid.

Examples of tackifiers are cellulose derivatives, starch derivatives, polyacrylates, polyvinyl pyrrolidone, and natural polymers such as alginates and gelatin.

Adjuvants may include spreading agents such as isopropyl myristate, dipropylene glycol pelargonate, silicone oils, fatty acid esters, triglycerides, fatty alcohols, mixture of ethyl nonafluoroisobutyl ether and ethyl nonafluorobutyl ether (Cosmetic Fluid CF-76, 3M), and mixture of methyl nonafluoroisobutyl ether and methyl nonafluorobutyl ether (Cosmetic Fluid CF-61, 3M). For example, pour-on and spot-on formulations may advantageously comprise carriers that assist rapid distribution over the surface of the skin and in the coat of the host animal and are generally termed spreading agents. Many spreading oil/solvent combinations are suitable, e.g., oily solutions; alcoholic and isopropanolic solutions, e.g., solutions of 2-octyl dodecanol or oleyl alcohol; solutions in esters of monocarboxylic acids, such as isopropyl myristate, isopropyl palmitate, lauric acid oxalic ester, oleic acid oleyl ester, oleic acid decyl ester, hexyl laurate, oleyl oleate, decyl oleate, caproic acid esters of saturated fatty alcohols of chain length C₁₂-C₁₈; solutions of esters of dicarboxylic acids, such as dibutyl phthalate, diisopropyl isophthalate, adipic acid diisopropyl ester, di-n-butyl adipate or solutions of esters of aliphatic acids, e.g., glycols. It may be advantageous for a dispersant known from the pharmaceutical or cosmetic industry also to be present. Examples are pyrrolidin-2-one, N-alkylpyrrolidin-2-one, acetone, polyethylene glycol and its ethers and esters, propylene glycol or synthetic triglycerides.

The formulations of the present invention may further include sorbitan monolaurate, dipropyleneglycol monomethyl ether, triethanolamine, benzyl alcohol, isopropyl alcohol, and/or ethyl acetoacetate.

In some embodiments, the veterinarily acceptable carrier may be in the form of an emulsion or solution for application to a localized region of the animal's skin (e.g. between the two shoulders as in spot-on type applications). Formulations may include solutions to be sprayed, poured, spread, or spotted onto the animal, oils, creams, ointments or any other appropriate fluid formulation for topical administration. Pour-on and spot-on formulations may be poured, spotted or sprayed onto limited areas of the skin, the active compound spreading on the body surface. Pour-on and spot-on formulations may be prepared by dissolving, suspending or emulsifying the active compound in suitable solvents or solvent mixtures which are tolerated by the skin. The formulation may be in the form of a ready-to-use solution that is applied topically and locally on the animal.

Veterinarily acceptable emulsions are either of the water-in-oil type or of the oil-in-water type. They are prepared by dissolving the fipronil either in the hydrophobic or in the hydrophilic phase and homogenizing this phase with the solvent of the other phase, with the aid of suitable emulsifiers and, if appropriate, other adjuvants such as colorants, spreading agents, absorption accelerators, preservatives, antioxidants, light stabilizers, and/or viscosity-increasing substances.

Examples of hydrophobic phase (oils) include paraffin oils, silicone oils, natural vegetable oils such as sesame seed oil, almond oil, castor oil, synthetic triglycerides such as caprylic/capric acid biglyceride, triglyceride mixture formed from vegetable fatty acids of chain length C8-12 or with other specifically selected natural fatty acids, partial glyceride mixtures of saturated or unsaturated fatty acids which may also contain hydroxyl groups, and mono- and diglycerides of the C8/C10-fatty acids. Fatty acid esters such as ethyl stearate, di-n-butyryl adipate, hexyl laurate, dipropylene glycol pelargonate, esters of a branched fatty acid of medium chain length with saturated fatty alcohols of chain length C16-C18, isopropyl myristate, isopropyl palmitate, caprylic/capric esters of saturated fatty alcohols of chain length C12-C18, isopropyl stearate, oleyl oleate, decyl oleate, ethyl oleate, ethyl lactate, waxy fatty acid esters such as dibutyl phthalate, diisopropyl adipate, ester mixtures related to the latter, and fatty alcohols such as isotridecyl alcohol, 2-octyldodecanol, cetylstearyl alcohol and oleyl alcohol.

Useful viscosity-increasing substances and substances which stabilize the emulsion include carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silica, or mixtures of the substances mentioned.

The formulations of the present invention include so called “pour-on” and “spot-on” formulations. The localized region may include, or may be wholly comprised of, a wound to the dermal layer, such as a cut or sore. Thus, in some embodiments, the present invention is useful in treating ectoparasite infection of a wound.

As noted above, the formulation is typically applied to a localized region that is less than 10% of the total surface area of the animal. In some embodiments, the localized region is less than 5% or 2% of the total surface area of the animal. Without being limited by any particular mechanism of action, it is believed that the fipronil diffuses beyond the localized region over the domestic animal's body. In some embodiments, the formulations of the present invention are applied by being deposited onto the skin using “spot-on” or “pour-on” applications. In some embodiments useful for cats and dogs, this application is localized over a surface area of less than 10 cm², especially of between 5 and 10 cm². In some embodiments, the formulation is applied at one or two points, typically between the animal's shoulders.

In some embodiments, the formulation is applied to the animal's back and at several points or along the line of the back, and applied in low volume, such as 5 to 20 ml per 100 kg, or 10 ml per 100 kg. In some embodiments, the total volume is from 0.075 ml to 150 ml per animal, sometimes 0.1 ml to 10 ml, often less than about 5 ml. For example, the volume applied to cats may be from about 0.3 to 1 ml for cats, and from about 0.1 to 5 ml for dogs, according to the weight of the animal.

Preferred formulations are sufficiently persistent to reduce the frequency and the cost associated with administration to the domestic animal. In some cases, the formulation is applied no more than twice per week, no more than once per week, or no more than once per month. Thus, certain formulations of the present invention maintain persistent efficacy for at least 48 hours, 1 week, 1 month, 2 months or in some cases up to 6 months. The formulations may also be sufficiently persistent to withstand washing of the domestic animal with an aqueous solution (e.g. soap and water). In some embodiments, the methods of the present invention include washing the animal with an aqueous solution after applying the formulation. Thus, the formulation may maintain persistent efficacy after at least one or 5 aqueous solution washes.

A variety of ectoparasites may be targeted using the formulations of the present invention. In some embodiments, the ectoparasite is a flea, fly, or louse, including flea eggs, flea larvae, fly eggs or fly larvae. Where the formulations of the present invention are designed to target flea eggs, flea larvae, fly eggs or fly larvae, the life-cycle of the flea and/or fly is broken thereby reducing environmental population pressures. For the purposes of the present invention, the term flea is understood to refer to all the usual or accidental species of parasitic flea of the order Siphonaptera, and in particular the genus Ctenocephalides, in particular the cat flea (C. felis) and dog flea (C. canis), rat flea (Xenopsylla cheopis) and human flea (Pulex irritans).

The local topical formulations may be prepared by simply mixing the constituents as defined above. For example, the active material is mixed in the veterinarily acceptable carrier and other components (if desired) are then added.

The dose and concentration of fipronil in the formulations of the present invention are chosen to optimize the efficacy and persistency of the formulations. In some embodiments, the concentration of fipronil in the formulation is at least 100 g/L, 150 g/L, or 200 g/L; or from 1 to 50% (w/v), 5 to 35% (w/v), or 10 to 20% (w/v). In some preferred embodiments, the concentration is about 200 g/L or 20% (w/v). In certain applications (e.g. where the domestic animal is a pet), the concentration is also chosen to minimize any undesired appearance (e.g. white crystals of fipronil) of the animal after application of the formulation. The total amount of fipronil administered to the domestic animal is typically from 1 to 50 mg per kg of body weight, 2 to 25 mg per kg of body weight, or 5 to 15 mg per kg of body weight.

### III. Examples

A study was conducted which was designed to observe the safety, appearance and potential for run-off of topical solutions of fipronil following application on dogs. The formulations applied to dogs were as follows: 10% (wt/vol) fipronil in dipropylene glycol monomethyl ether (q.s.), 10% (wt/vol) fipronil in propylene carbonate (q.s.), and 10% (wt/vol) fipronil in N,N-dimethylacetamide (q.s.). A total of 12 dogs were included in the study (four/group). All dogs were treated on Day 0 with the appropriate test formulation at a dose volume of 0.075 mL/kg according to their individual bodyweights. Post treatment assessments were done at 5, 15 and 30 minutes, and 1, 3 and 24 hours. All formulations were safe when applied on dogs. There were no general adverse reactions nor were there any negative effects on skin or hair. All three formulations had acceptable appearance upon drying.

A spot-on solution containing a spreading agent for topical administration to dogs for treatment or prevention of tick and/or flea infestation may contain the following ingredients:
fipronil: 10%
Cosmetic Fluid CF-76: 20%
isopropyl alcohol: 20%
dipropylene glycol monomethyl ether: q.s. to 100%

1. A local topical formulation comprising an ectoparasitically effective amount of fipronil and a veterinarily acceptable carrier, in the absence of an effective amount of a fipronil crystallization inhibitor.

2. The formulation of claim 1, wherein said fipronil is present at a concentration of about 10% (w/v).

3. The formulation of claim 2, wherein said veterinarily acceptable carrier is dipropylene glycol monomethyl ether.

4. The formulation of claim 2, wherein said veterinarily acceptable carrier is propylene carbonate.

5. The formulation of claim 2, wherein said veterinarily acceptable carrier is N,N dimethylacetamide.

6. The formulation of claim 1, further comprising a colorant, an antioxidant, a light stabilizer, or a combination thereof.

7. The formulation of claim 1, further comprising a second active ingredient.

8. A local topical formulation consisting essentially of an ectoparasitically effective amount of fipronil and a veterinarily acceptable carrier.

9. The formulation of claim 8, wherein said fipronil is present at a concentration of about 10% (w/v).

10. The formulation of claim 8, wherein said veterinarily acceptable carrier is dipropylene glycol monomethyl ether.

11. The formulation of claim 8, wherein said veterinarily acceptable carrier is propylene carbonate.

12. The formulation of claim 8, wherein said veterinarily acceptable carrier is N,N dimethylacetamide.

13. The formulation of claim 8, that further includes a colorant, an antioxidant, a light stabilizer, or a combination thereof.

14. A method for control of an ectoparasite on a domestic animal, said method comprising topically applying to a localized region having a surface area of less than or equal to 10% of the total surface area of said domestic animal the formulation of claim 1.

15. The method of claim 14, wherein said formulation is applied to a localized region that is less than 5% of the total surface area of said animal.

16. The method of claim 14, wherein said formulation is applied to a localized region that is less than 2% of the total surface area of said animal.

17. The method of claim 14, wherein said formulation is applied no more than twice per week.

18. The method of claim 14, wherein the ectoparasite is a flea, tick, fly, or louse.

19. The method of claim 14, wherein the ectoparasite is a flea.

20. The method of claim 14, wherein said domestic animal is a mammal.

21. The method of claim 20, wherein said mammal is a canine or feline.
